(19) United States
(12) Patent Application Publication
(10) Pub. No.: US 2022/0176007 A1
(43) Pub. Date: Jun. 9, 2022

(54) AIR PURIFIER AND PRODUCTION METHOD FOR AIR PURIFIER

(71) Applicant: ECLAIR, LTD., Tokyo (JP)

(72) Inventor: Masakazu GOTO, Tokyo (JP)

(21) Appl. No.: 17/602,570

(22) PCT Filed: Apr. 20, 2020
(86) PCT No.: PCT/JP2020/017071

§ 371 Date: Oct. 8, 2021

(30) Foreign Application Priority Data
Apr. 23, 2019 (JP) 2019-081771

(51) Int. Cl. A61L 9/22; H01T 23/00; H01T 19/00; F24F 8/20

(52) U.S. Cl. CPC A61L 9/22; F24F 8/20; H01T 19/00; H01T 23/00

(57) ABSTRACT

An air purifier that enables reduction of production processes and is easy to produce, and a production method for the air purifier are proposed. An air purifier (1) includes: a first electrode-plate (100) made of a conductive plate and including a first electrode portion (10) that is plate-like and includes a plurality of first electrode-structures (12), the first electrode portion including a first conductive area (18) between a first hollow (14) and second hollows (16), a second electrode portion (20) that is plate-like and includes a plurality of second electrode-structures (22), the second electrode portion including a second conductive area (28) between a third hollow (24) that is larger in diameter than the first hollow, and fourth hollows (26), and a coupling portion (50) having a bent shape to place the second electrode portion vertically above the first electrode portion; and a second electrode-plate (200) made of a conductive plate and located separate from the first electrode-structures and the second electrode-structures, a plurality of third electrode-structures (32) that extends toward the first electrode-structures and the second electrode-structures being formed integrally in the second electrode-plate.

### TECHNICAL FIELD

The present invention relates to an air purifier that generates an ionic wind, and a production method for the air purifier. The present invention relates particularly to an air purifier that includes no fan and generates an ionic wind, and a production method for the air purifier.

### BACKGROUND ART

Devices are conventionally known that generate an ionic wind by a corona discharge. A conventional device includes a first metal layer having a first pattern of hollows, and a second metal layer having a second pattern of hollows that differs from the first pattern of hollows, the second metal layer being placed vertically above the first metal layer with a gap therebetween. The device supplies a first voltage to the first and second metal layers to generate a potential difference between the first and second metal layers and a rod-like electrode located at the central axis of the first and second patterns of hollows to thereby cause a corona discharge and produce an ionic wind. While the device generates an ionic wind by a corona discharge, a desire exists for an increase in volume of the ionic wind, and as such, a device that includes a plurality of the electrodes is also known.

In an air purifier as described above, the second metal layer is placed vertically above the first metal layer in such a manner that the first pattern of hollows and the second pattern of hollows are concentric. For placing the second metal layer above the first metal layer, the first metal layer and the second metal layer are secured with pins or the like with spacers therebetween (see Patent Literature 1). This assembly work, however, increases the production processes, inhibiting reduction of production time and costs. Concerning the rod-like electrode located at the central axis of the first and second patters of hollows, Patent Literature 1 proposes to facilitate production by providing a flat plate structure at portions of rod-like electrodes remote from the first and second patterns of hollows and by supporting the flat plate structure with a flat plate serving as a base.

### Prior-Art Publication

### Patent Literature

Patent Literature 1:
   Japanese Registered Utility Model No. 3210591

### SUMMARY OF INVENTION

### Technical Problem

It is, however, desired that the production processes be further reduced in order to mass-produce air purifiers and to reduce costs. Thus, the present invention proposes an air purifier that enables reduction of production processes and is easy to produce, and a production method for the air purifier.

### Solution to Problem

As a solution for the problems described above, as shown in, for example, FIGS. 1 and 2, an air purifier 1 according to a first aspect of the present invention includes: a first electrode-plate 100 made of a conductive plate, the first electrode-plate 100 including, a first electrode portion 10 that is plate-like, the first electrode portion 10 including a plurality of first electrode-structures 12, each of the first electrode-structures 12 having a first hollow 14 that is substantially shaped as a circle, and second hollows 16 that are substantially shaped as a circular ring that is concentric with the first hollow 14, and having a first conductive area 18 substantially shaped as a circular ring between the first hollow 14 and the second hollows 16, a second electrode portion 20 that is plate-like, the second electrode portion 20 including a plurality of second electrode-structures 22, each of the second electrode-structures 22 having a third hollow 24 that is substantially shaped as a circle and is larger in diameter than the first hollow 14, and fourth hollows 26 that are substantially shaped as a circular ring that is concentric with the third hollow 24, and having a second conductive area 28 substantially shaped as a circular ring between the third hollow 24 and the fourth hollows 26, the second electrode-structures 22 being formed at locations corresponding to respective locations of the first electrode-structures 12, and a coupling portion 50 coupling the first electrode portion 10 and the second electrode portion 20 and having a bent shape to place the second electrode portion 20 vertically above the first electrode portion 10 such that each of the first electrode-structures 12 and a corresponding one of the second electrode-structures 22 are concentric; and a second electrode-plate 200 made of a conductive plate, the second electrode-plate 200 being located separate from the first electrode-structures 12 and the second electrode-structures 22, a plurality of third electrode-structures 32 being formed integrally in the second electrode-plate 200, each of the third electrode-structures 32 extending along a central axis of a corresponding one of the first electrode-structures 12 and a corresponding one of the second electrode-structures 22, toward the corresponding one of the first electrode-structures 12 and the corresponding one of the second electrode-structures 22, wherein the first electrode-structures 12, the second electrode-structures 22, and the third electrode-structures 32 are arranged in this order, and by application of a negative voltage to the first electrode-plate 100 and a positive voltage to the second electrode-plate 200, corona discharges are caused between the first electrode-structures 12 and the third electrode-structures 32 and between the second electrode-structures 22 and the third electrode-structures 32 to generate an ionic wind F that flows from the first electrode-structures 12 and the second electrode-structures 22 in a direction away from the third electrode-structures 32.

In this configuration, corona discharges are caused between the first electrode-structures and the third electrode-structures and between the second electrode-structures and the third electrode-structures to generate an ionic wind that flows from the first electrode-structures and the second electrode-structures 22 in a direction away from the third electrode-structures 32, whereby a large volume of the ionic wind can be generated. Furthermore, since the first electrode portion including the first electrode-structures, the second electrode portion including the second electrode-structures, and a coupling portion having a bent shape to place the second electrode portion vertically above the first electrode portion are formed in the first electrode-plate made of a conductive plate, the air purifier is easy to produce with reduced production processes.

In an air purifier 1 according to a second aspect of the present invention, an end 33 of each of the third electrode-structures 32 is shaped as a plane, the end 33 being located toward the corresponding one of the second electrode-structures 22, as shown in, for example, FIG. 1 or 5. In this configuration, a touch on the end of the third electrode-structure during maintenance work or the like is safe because the end is shaped as a plane.

In an air purifier 1 according to a third aspect of the present invention, a distance L1 between the first conductive area 18 of one of the first electrode-structures 12 and a corresponding one of the third electrode-structures 32 is longer than a distance L2 between the second conductive area 28 of a corresponding one of the second electrode-structures 22 and the corresponding one of the third electrode-structures 32, as shown in, for example, FIG. 1. In this configuration, since the distance from the corresponding one of the third electrode-structures to the corresponding one of the first electrode-structures having a small diameter and thus causing the ionic wind to be in a high density is longer than the distance from the corresponding one of the third electrode-structures to the corresponding one of the second electrode-structures having a large diameter and thus causing the ionic wind to be in a low density, and thus a corona discharge in the first electrode-structures is weaker than in the second electrode-structures, and an ionic wind with uniform densities as a whole can be emitted. Note that the distance between the first conductive area and the corresponding one of the third electrode-structures refers to the shortest distance therebetween, and that the distance between the second conductive area and the corresponding one of the third electrode-structures refers to the shortest distance therebetween.

In an air purifier 1 according to a fourth aspect of the present invention, a width of the coupling portion 50 is smaller than a width of the first electrode portion 10 and a width of the second electrode portion 20 as shown in, for example, FIG. 2. In this configuration, a process in which the conductive plate for the first electrode portion is folded to place the second electrode portion vertically above the first electrode portion is facilitated, and also the first electrode-plate can be reduced in weight. Note that the widths of the first electrode portion, the second electrode portion, and the coupling portion refer to lengths of the first electrode portion, the coupling portion, and the second electrode portion before the folding of the first electrode-plate, in a direction orthogonal to the direction in which the first electrode portion, the coupling portion, and the second electrode portion are aligned, and they are the lengths in an up-and-down direction in FIG. 2.

In an air purifier 1 according to a fifth aspect of the present invention, as shown in, for example, FIGS. 1 and 2, the first electrode-plate 100 further includes a fourth electrode portion 40 that is plate-like, the fourth electrode portion 40 including a plurality of fourth electrode-structures 42, each of the fourth electrode-structures 42 having a fifth hollow 44 that is substantially shaped as a circle and is larger in diameter than the third hollow 24, and having a fourth conductive area 48 that is a conductive area at a rim of the fifth hollow 44, the fourth electrode-structures 42 being formed at locations corresponding to respective locations of the second electrode-structures 22; and a second coupling portion 52 coupling the fourth electrode portion 40 and the second electrode portion 20 and having a bent shape to place the fourth electrode portion 40 vertically above the second electrode portion 20 such that each of the fourth electrode-structures 42 and a corresponding one of the second electrode-structures 22 are concentric. In this configuration, corona discharges are also caused between the fourth electrode-structures and the third electrode-structures, in addition to those caused between the first electrode-structures and the third electrode-structures and between the second electrode-structures and the third electrode-structures, to generate an ionic wind that flows from the fourth electrode-structures in a direction away from the third electrode-structures, whereby a large volume of the ionic wind can be generated. Since the first electrode portion including the first electrode-structures, the second electrode portion including the second electrode-structures, the coupling portion having a bent shape to place the second electrode portion vertically above the first electrode portion, the fourth electrode portion including the fourth electrode-structures, and the second coupling portion having a bent shape to place the second electrode portion vertically above the fourth electrode portion are formed in the first electrode-plate made of a conductive plate, the air purifier is easy to produce with reduced production processes.

In a production method for an air purifier according to a sixth aspect of the present invention, which is a production method for the air purifier 1 of any of the first to fourth aspects as shown in, for example, FIGS. 2 to 4, the first electrode portion 10, the second electrode portion 20, and the coupling portion 50 are formed in a conductive plate 110 that is plate-like, by performing press working, and bending is performed on the coupling portion 50 of the conductive plate 110 in which the first electrode portion 10, the second electrode portion 20, and the coupling portion 50 have been formed, to produce the first electrode-plate 100. In this configuration, the first electrode-plate can be produced by performing the press working and the bending, and thus, the production method for the air purifier enables the air purifier to be produced easily with reduced production processes.

In a production method for the air purifier according to a seventh aspect of the present invention, the third electrode-structures 32 are formed in a conductive plate 210 that is plate-like, by performing press working as shown in, for example, FIG. 5. In this configuration, the second electrode-plate can be produced by performing the press working, and thus, the production method for the air purifier enables the air purifier to be produced easily with reduced production processes.

In a production method for an air purifier according to an eighth aspect of the present invention, which is a production method for the air purifier of the fifth aspect as shown in, for example, FIGS. 2 to 4, the first electrode portion 10, the second electrode portion 20, the fourth electrode portion 40, the coupling portion 50, and the second coupling portion 52 are formed in a conductive plate 110 that is plate-like, by performing press working, and bending is performed on the coupling portion 50 and the second coupling portion 52 of the conductive plate 110 in which the first electrode portion 10, the second electrode portion 20, the fourth electrode portion 40, the coupling portion 50, and the second coupling portion 52 have been formed, to produce the first electrode-plate 100. In this configuration, the first electrode-plate can be produced by performing the press working and the bending, and thus, the production method for the air purifier enables the air purifier to be produced easily with reduced production processes.

In a production method for the air purifier according to a ninth aspect of the present invention, the third electrode-structures 32 are formed in a conductive plate 210 that is plate-like, by performing press working as shown in, for example, FIG. 5. In this configuration, the second electrode-plate can be produced by performing the press working, and thus, the production method for the air purifier enables the air purifier to be produced easily with reduced production processes.

### Advantageous Effects of Invention

The air purifier according to the present invention can generate a large volume of an ionic wind and is easy to produce with a small number of production processes. The production method for the air purifier according to the present invention enables the air purifier to be produced easily with a small number of production processes.

The basic Japanese patent application No. 2019-081771, filed Apr. 23, 2019, is hereby incorporated by reference in its entirety in the present application.

The present invention will become more fully understood from the detailed description given below. However, the detailed description and the specific embodiments are only illustrations of the desired embodiments of the present invention, and so are given only for an explanation. Various possible changes and modifications will be apparent to those of ordinary skill in the art on the basis of the detailed description.

The applicant has no intention to dedicate to the public any disclosed embodiment. Among the disclosed changes and modifications, those which may not literally fall within the scope of the present claims constitute, therefore, a part of the present invention in the sense of the doctrine of equivalents.

The use of the articles “a,” “an,” and “the” and similar referents in the specification and claims are to be construed to cover both the singular and the plural form of a noun, unless otherwise indicated herein or clearly contradicted by the context. The use of any and all examples, or exemplary language (e.g., “such as”) provided herein is intended merely to better illustrate the invention, and so does not limit the scope of the invention, unless otherwise stated.

### BRIEF DESCRIPTION OF DRAWINGS

FIG. 1 is a side view of a major part of an air purifier in an embodiment of the present invention.

FIG. 2 is a plan view of a conductive plate subjected to press working for a first electrode-plate.

FIG. 3 is a diagram of a process of bending being performed on the conductive plate subjected to the press working for the first electrode-plate, in which FIG. 3(a) is a plan view, and FIG. 3(b) is a side view.

FIG. 4 is a diagram of the first electrode-plate subjected to the bending, in which FIG. 4(a) is a plan view, and FIG. 4(b) is a side view.

FIG. 5 is a plan view of a conductive plate subjected to press working for a second electrode-plate.

FIG. 6 is a five-side view of the second electrode-plate subjected to bending, in which FIG. 6(a) is a plan view, FIG. 6(b) is a front view, FIG. 6(c) is a right side view, FIG. 6(d) is a back view, and FIG. 6(e) is a left side view.

### DESCRIPTION OF EMBODIMENT

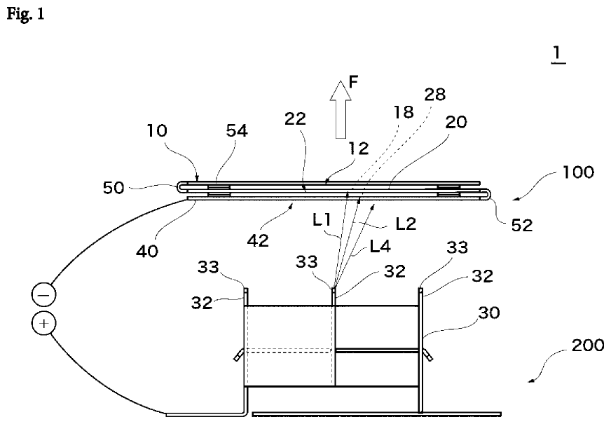

An embodiment of the present invention is described below with reference to the drawings. Elements that are identical or corresponding to each other are denoted by the same signs in the drawings, and duplicated description is omitted. FIG. 1 is a sectional view of a major part of an air purifier 1. In the major part, the air purifier 1 includes a first electrode-plate 100 including a first electrode portion 10 that is plate-like, a second electrode portion 20 that is plate-like, and a fourth electrode portion 40 that is plate-like, the first electrode portion 10, the second electrode portion 20, and the fourth electrode portion 40 being placed vertically one above the other; and a second electrode-plate 200 including third electrode-structures 32 and located opposite the first, second, and fourth electrode portions. Although not shown in the drawings, the air purifier 1 includes a power supply for applying a negative voltage to the first electrode-plate 100 and a positive voltage to the second electrode-plate 200, a casing that secures and houses these members, takes in the outside air and discharges an ionic wind, and a switch to activate and deactivate the air purifier 1. The air purifier 1 discharges an ionic wind generated at the major part through a nozzle formed in the casing.

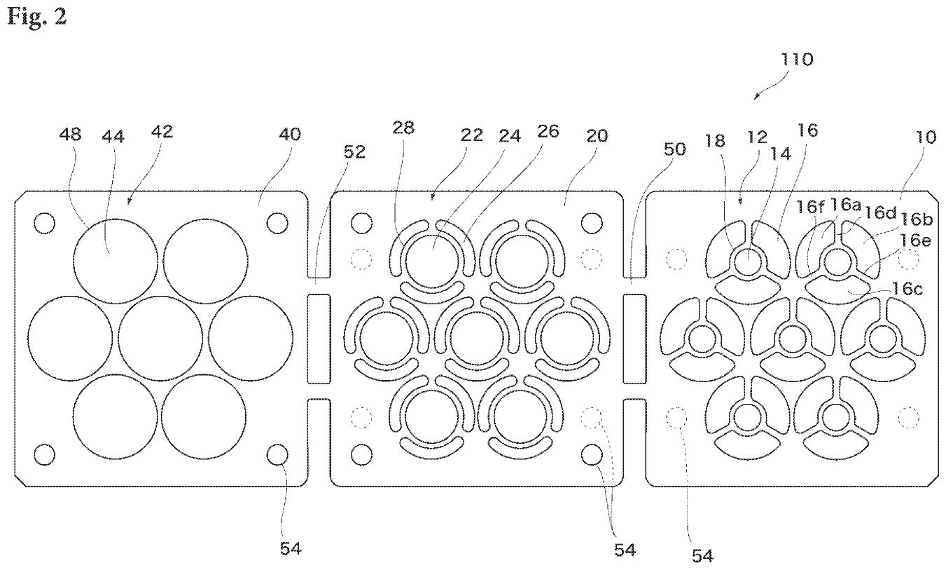

FIG. 2 is a plan view of a conductive plate 110 subjected to press working for the first electrode-plate 100. The conductive plate 110 is typically a metal plate of iron, copper, aluminum, or the like, but this is not a limitation. The conductive plate 110 has been subjected to the press working to form the first electrode portion 10, the second electrode portion 20, the fourth electrode portion 40, a coupling portion 50 coupling the first electrode portion 10 and the second electrode portion 20, and a second coupling portion 52 coupling the second electrode portion 20 and the fourth electrode portion 40.

In the first electrode portion 10, a plurality of first electrode-structures 12 is formed. Specifically, the first electrode-structure 12 is formed as described below. A first hollow 14 that is substantially shaped as a circle and second hollows 16 that are substantially shaped as a circular ring are formed concentrically. If the first hollow 14 that is substantially shaped as a circle is shaped as a circle, the distances from the third electrode-structure 32 are uniform, so that a uniform corona discharge can be obtained, and thus the ionic wind is also uniform. However, the first hollow 14 that is substantially shaped as a circle may be shaped as a polygon, an ellipse, an onigiri-shape, or the like, as long as a corona discharge can be obtained. The second hollows 16 that are substantially shaped as a circular ring are formed concentric with the first hollow 14 such that a first conductive area 18 is formed around the first hollow 14. Here, the wording “concentric” does not require strict concentricity and allows misalignment to the extent that the first conductive area 18 is formed. The second hollows 16 that are substantially shaped as a circular ring include, for example, three segment-shaped hollows 16a, 16b, and 16c and connecting parts 16d, 16e, and 16f that are parts located between the segment-shaped hollows 16a, 16b, and 16c to support the first conductive area 18. The number of the segment-shaped hollows and the number of the connecting parts are not limited to three, and may be two, or four or more. In this fashion, the first conductive area 18 that is substantially shaped as a circular ring is formed between the first hollow 14 and the second hollows 16. The first conductive area 18, in particular its inner edge, causes a corona discharge between the inner edge and the third electrode-structure 32.

The first electrode portion 10 includes seven first electrode-structures 12 in a plate. When a plurality of first electrode-structures 12 is provided as in this case, many corona discharges are caused and thus a large volume of the ionic wind can be generated. The seven first electrode-structures 12 are located at equal distances from each other. Such placement can provide a uniform ionic wind. The number of first electrode-structures 12 is not limited to seven and may be arbitrarily changed in accordance with a required volume of the ionic wind. Additionally, the location of the first electrode-structures 12 at equal distances is not a limitation.

The second electrode portion 20 has a structure similar to that of the first electrode portion 10; thus, the differences are described with duplicate explanation omitted. In the second electrode portion 20, a plurality of second electrode-structures 22 is formed in a plate. Specifically, the second electrode-structure 22 is formed as described below. A third hollow 24 that is substantially shaped as a circle and corresponds to the first hollow 14, and fourth hollows 26 that are substantially shaped as a circular ring and correspond to the second hollows 16 are formed concentrically, so that a second conductive area 28 is formed. Note that the third hollow 24 is larger in diameter than the first hollow 14. That is, the second electrode-structure 22 has a larger diameter than the first electrode-structure 12 does. As in the case with the second hollows 16, the fourth hollows 26 that are substantially shaped as a circular ring include three segment-shaped hollows and connecting parts that are parts located between the segment-shaped hollows to support the second conductive area 28. The outside diameter of the fourth hollows 26 is the same as that of the second hollows 16, but this is not a limitation. Providing the same outside diameters for the fourth hollows 26 and the second hollows 16 can form a smooth pathway for an ionic wind generated by a corona discharge. The second electrode-structures 22 are formed at locations corresponding to respective locations of the first electrode-structures 12. Here, the wording “locations corresponding” means that each of the second electrode-structures 22 and a corresponding one of the first electrode-structures 12 have such a positional relationship as to be concentric when the conductive plate 110 is folded to provide the first electrode-plate 100.

The fourth electrode portion 40 has a structure similar to those of the first electrode portion 10 and the second electrode portion 20; thus, the differences are described with duplicate explanation omitted. In the fourth electrode portion 40, a plurality of fourth electrode-structures 42 is formed in a plate. Specifically, the fourth electrode-structure 42 is formed as described below. A fifth hollow 44 that is substantially shaped as a circle is formed. The fifth hollow 44 is larger in diameter than the third hollow 24. A conductive area at the rim of the fifth hollow 44 is the fourth conductive area 48. That is, the fourth electrode-structure 42 has a larger diameter than the second electrode-structure 22 does. The outside diameter of the fifth hollow 44 is the same as those of the second hollow 16 and the fourth hollow 26, but this is not a limitation. Providing the same outside diameters for the fifth hollow 44, the second hollow 16, and the fourth hollow 26 can form a smooth pathway for an ionic wind generated by a corona discharge.

The coupling portion 50 is located between the first electrode portion 10 and the second electrode portion 20 for connection thereof. The coupling portion 50 is a portion to be subjected to bending so that the second electrode portion 20 is placed vertically above the first electrode portion 10 to form the first electrode-plate 100. Thus, the coupling portion 50 may have a length sufficient to be bent to place the second electrode portion 20 vertically above the first electrode portion 10. Also, the coupling portion 50 may have a width smaller than those of the first electrode portion 10 and the second electrode portion 20. The reduced width of the coupling portion 50 leads

to a reduced force required for the bending, thus facilitating the bending. Here, the term “length” refers to a length in a direction in which the first electrode portion 10, the coupling portion 50, and the second electrode portion 20 are aligned (a horizontal direction in FIG. 2), and the term “width” refers to a length in a direction orthogonal to the direction in which the first electrode portion 10, the coupling portion 50, and the second electrode portion 20 are aligned (an up-and-down direction in FIG. 2). The coupling portion 50 is normally made of the same material as those of the first electrode portion 10 and the second electrode portion 20 but may be made of a different material.

The second coupling portion 52 is located between the second electrode portion 20 and the fourth electrode portion 40 for connection thereof. Other aspects are similar to those of the coupling portion 50 and thus duplicated description is omitted.

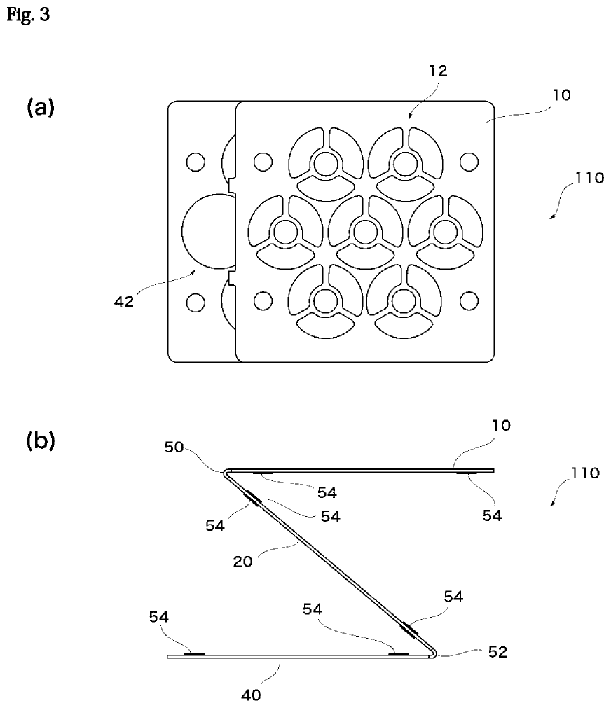

FIG. 3 illustrates a state in which the coupling portion 50 and the second coupling portion 52 are being subjected to bending so that the first electrode portion 10, the second electrode portion 20, and the fourth electrode portion 40 are placed vertically one above the other. A surface of the first electrode portion 10 facing the second electrode portion 20, a surface of the second electrode portion 20 facing the first electrode portion 10, a surface of the second electrode portion 20 facing the fourth electrode portion 40, and a surface of the fourth electrode portion 40 facing the second electrode portion 20 each have four spacers 54 affixed thereto. The spacers 54 determine the sizes of gaps between the electrode portions 10, 20, and 40, allowing the gaps to remain constant. Spacer dents (not shown) are normally formed in the conductive plate 110 during the press working, and the spacers 54 are fitted into the respective spacer dents for affixation. The material of the spacers 54 is preferably the same as that of the conductive plate 110 for enhanced processability. The material of the spacers 54, however, may be different from that of the conductive plate 110 and may be a conductor or an insulator.

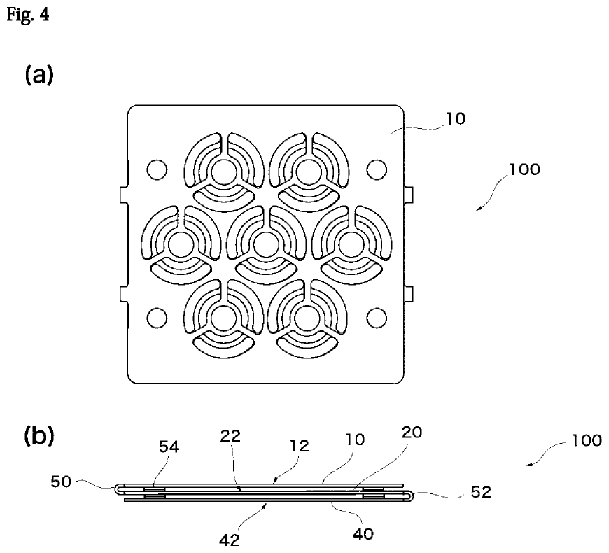

FIG. 4 illustrates a state in which the bending is finished and the first electrode portion 10, the second electrode portion 20, and the fourth electrode portion 40 are placed vertically one above the other. The spacers 54 of the first electrode portion 10 are in contact with the corresponding spacers 54 of the second electrode portion 20, and the spacers 54 of the fourth electrode portion 40 are in contact with the corresponding spacers 54 of the second electrode portion 20, whereby the gaps are created between the electrode portions 10, 20, and 40. The first electrode-plate 100 can be produced by performing the press working on a single conductive plate and by performing the bending on the conductive plate 110 subjected to the press working as described above; thus, the first electrode-plate 100 can be produced with ease. Furthermore, the production through the press working and the bending can lead to reduced misalignment between each of the first electrode-structures 12, the corresponding one of the second electrode-structures 22, and the corresponding one of the fourth electrode-structures 42, which are to be concentric, in comparison with the assembly work in which a plurality of metal layers is secured with pins or the like with spacers therebetween.

A configuration of the second electrode-plate 200 is described below with reference to FIGS. 1, 5, and 6. The second electrode-plate 200 is located opposite the first electrode portion 10, the second electrode portion 20, and the fourth electrode portion 40 and serves as a third electrode portion 30 that generates corona discharges between the third electrode portion 30 and the first, second, and fourth electrode portions. The third electrode portion 30 includes the third electrode-structures 32. Each of the third electrode-structures 32 is rod-like and extends along a central axis of a corresponding one of the first electrode-structures 12, a corresponding one of the second electrode-structures 22, and a corresponding one of the fourth electrode-structures 42. The term “rod-like” does not necessarily mean an elongate element having a constant diameter. The third electrode-structure 32 may be a plate tapered to a narrower end, or the like, or may have any shape as long as it can generate a corona discharge from its end. An end 33 of the third electrode-structure 32, which is an end of the third electrode-structure 32 located toward the first electrode portion 10, the second electrode portion 20, and the fourth electrode portion 40, is shaped as a plane. The term “plane” may include a shape in which the end 33 has its perimeter rounded or chamfered, or the end 33 is shaped as a smooth spheroid (a solid of revolution about a single axis) as a whole.

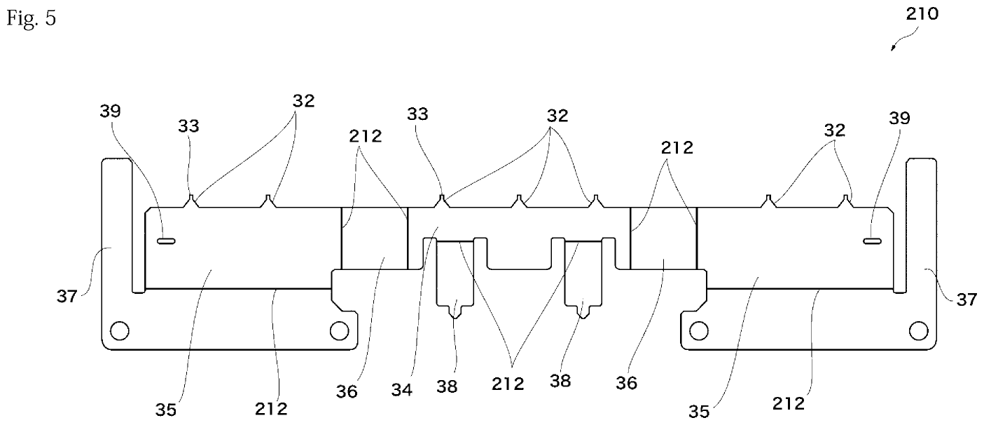

FIG. 5 is a plan view of a conductive plate 210 subjected to press working for the second electrode-plate 200. The conductive plate 210 includes bend lines 212 provided in such a manner that the conductive plate 210 can be bent into a solid in which each of the seven third electrode-structures 32 is located concentric with a corresponding one of the seven first electrode-structures 12, a corresponding one of the seven second electrode-structures 22, and a corresponding one of the seven fourth electrode-structures 42 formed in the first electrode-plate 100. The conductive plate 210 is segmented by the bend lines 212 into a middle portion 34 including three third electrode-structures 32, two side portions 35 each including two third electrode-structures 32, two linking portions 36 each linking the middle portion 34 and a corresponding one of the side portions 35, bottom surface portions 37 serving as a base for the second electrode-plate 200, and brace portions 38 serving as braces between the middle portion 34 and the side portions 35. Each of the side portions 35 has a through-hole 39 in which an end of a corresponding one of the brace portions 38 is to be inserted.

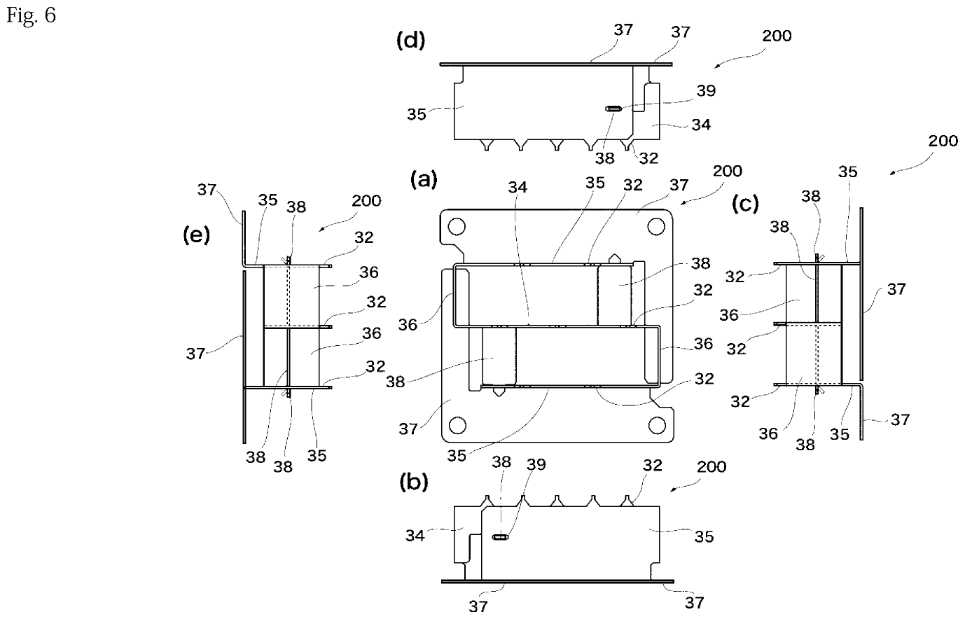

FIG. 6 is a five-side view of the second electrode-plate 200, which is fabricated from the conductive plate 210. FIG. 6(a) is a plan view, FIG. 6(b) is a front view, FIG. 6(c) is a right side view, FIG. 6(d) is a back view, and FIG. 6(e) is a left side view. The conductive plate 210 is bent along the bend lines 212, and the ends of the brace portions 38 are inserted through the respective through-holes 39. Bending the inserted portions of the brace portions 38 as shown by broken lines in FIG. 6 stabilizes the shape of the second electrode-plate 200. The second electrode-plate 200 can be produced by performing the press working on a single conductive plate and by bending the conductive plate 210 subjected to the press working as described above; thus, the second electrode-plate 200 can be produced with ease. Furthermore, the production through the press working and the bending can lead to reduced production processes for the second electrode-plate 200, in comparison with the assembly work in which the flat plate structure is provided at the remote base portions of the rod-like electrodes and the flat plate serving as a base is assembled to support the flat plate structure.

The first electrode-plate 100 and the second electrode-plate 200, which have been produced as described above, are secured to and housed in the casing and connected to the power supply to produce the air purifier 1.

Although the first electrode-plate 100 has been described as including the first electrode portion 10, the second electrode portion 20, and the fourth electrode portion 40, the first electrode-plate 100 may include no fourth electrode portion 40. Alternatively, the first electrode-plate 100 may further include a similar electrode portion.

Although the first electrode-plate 100 and the second electrode-plate 200 have been described as being formed through press working, only one of the first electrode-plate 100 and the second electrode-plate 200 may be formed through press working. In such cases, the production method will still be easier with fewer production processes than the conventional production method.

Although the coupling portion 50 and the second coupling portion 52 have been described as having widths smaller than those of the first electrode portion 10, the second electrode portion 20, and the fourth electrode portion 40, one or both of the coupling portion 50 and the second coupling portion 52 may have a width similar to those of the first electrode portion 10, the second electrode portion 20, and the fourth electrode portion 40.

The operation of the air purifier 1 is described next below. By application of a negative voltage to the first electrode portion 10, the second electrode portion 20, and the fourth electrode portion 40 and a positive voltage to the third electrode portion 30, corona discharges are caused between the third electrode-structures 32 and the first electrode-structures 12, between the third electrode-structures 32 and the second electrode-structures 22, and between the third electrode-structures 32 and the fourth electrode-structures 42. Potential difference applied varies with spacing between electrode structures and the sizes of the electrode structures.

Due to the corona discharges, an ionic wind is generated from the first electrode-structures 12, the second electrode-structures 22, and the fourth electrode-structures 42, in a direction away from the third electrode-structures 32 (upward in FIG. 1). That is, air is positively ionized at the first electrode-structures 12, the second electrode-structures 22, and the fourth electrode-structures 42; thus, the ionic wind is generated in a direction away from the third electrode-structures 32, to which the positive voltage is applied. An ionic wind is generated at the first electrode-structure 12, the second electrode-structure 22, and the fourth electrode-structure 42, that is, at three positions; thus, a large volume of the ionic wind flows.

A distance L1 between one of the first electrode-structures 12 and a corresponding one of the third electrode-structures 32 is longer than a distance L2 between a corresponding one of the second electrode-structures 22 and the corresponding one of the third electrode-structures 32. A distance L4 between a corresponding one of the fourth electrode-structures 42 and the corresponding one of the third electrode-structures 32 is shorter than the distance L2 between the corresponding one of the second electrode-structures 22 and the corresponding one of the third electrode-structures 32. The distance L4 refers to the shortest distance between the corresponding one of the fourth electrode-structures 42 and the corresponding one of the third electrode-structures 32. At the first electrode-structure 12, which has a small diameter, an ionic wind with a high density is generated. At the second electrode-structure 22, which has a larger diameter, an ionic wind with a lower density is generated. At the fourth electrode-structure 42, which has an even larger diameter, an ionic wind with an even lower density is generated. A longer distance from the third electrode-structure 32, however, leads to a weaker corona discharge, thus resulting in an ionic wind with a lower density. Thus, setting the distances to satisfy L1>L2>L4 tends to generate an ionic wind with uniform densities as a whole. In particular, each of the first electrode-structures 12, a corresponding one of the second electrode-structures 22, and a corresponding one of the fourth electrode-structures 42 are preferably placed on a paraboloid centering about a corresponding one of the third electrode-structure 32. The placement on a paraboloid tends to generate an ionic wind with uniform densities. The relation of the distances L1, L2, and L4 is not limited to the relation described above; the distances L1, L2, and L4 may have other relation or may be the same.

### INDUSTRIAL APPLICABILITY

The air purifier according to the present invention generates an ionic wind, thereby capable of decomposing viruses and chemical substances floating in spaces such as a room space, a vehicle interior space, a container space, and the like for sterilization and deodorization of the air.

Principal reference signs used in the present specification and the drawings are listed below.
1 air purifier
10 first electrode portion
12 first electrode-structure
14 first hollow
16 second hollow
18 first conductive area
20 second electrode portion
22 second electrode-structure
24 third hollow
26 fourth hollow
28 second conductive area
30 third electrode portion
32 third electrode-structure
33 end of third electrode-structure (end located toward second electrode-structure)
34 middle portion
35 side portion
36 linking portion
37 bottom surface portion
38 brace portion
39 through-hole
40 fourth electrode portion
42 fourth electrode-structure
44 fifth hollow
48 fourth conductive area
50 coupling portion
52 second coupling portion
54 spacer
100 first electrode-plate
110 conductive plate that is plate-like
200 second electrode-plate
F ionic wind
L1 distance between first electrode-structure and third electrode-structure
L2 distance between second electrode-structure and third electrode-structure
L4 distance between fourth electrode-structure and third electrode-structure

1. An air purifier comprising:
a first electrode-plate made of a conductive plate, the first electrode-plate comprising
a first electrode portion that is plate-like, the first electrode portion comprising a plurality of first electrode-structures, each of the first electrode-structures having a first hollow that is substantially shaped as a circle, and second hollows that are substantially shaped as a circular ring that is concentric with the first hollow, and having a first conductive area substantially shaped as a circular ring between the first hollow and the second hollows,
a second electrode portion that is plate-like, the second electrode portion comprising a plurality of second electrode-structures, each of the second electrode-structures having a third hollow that is substantially shaped as a circle and is larger in diameter than the first hollow, and fourth hollows that are substantially shaped as a circular ring that is concentric with the third hollow, and having a second conductive area substantially shaped as a circular ring between the third hollow and the fourth hollows, the second electrode-structures being formed at locations corresponding to respective locations of the first electrode-structures, and
a coupling portion coupling the first electrode portion and the second electrode portion and having a bent shape to place the second electrode portion vertically above the first electrode portion such that each of the first electrode-structures and a corresponding one of the second electrode-structures are concentric; and
a second electrode-plate made of a conductive plate, the second electrode-plate being located separate from the first electrode-structures and the second electrode-structures, a plurality of third electrode-structures being formed integrally in the second electrode-plate, each of the third electrode-structures extending along a central axis of a corresponding one of the first electrode-structures and a corresponding one of the second electrode-structures, toward the corresponding one of the first electrode-structures and the corresponding one of the second electrode-structures,
wherein the first electrode-structures, the second electrode-structures, and the third electrode-structures are arranged in this order, and
by application of a negative voltage to the first electrode-plate and a positive voltage to the second electrode-plate, corona discharges are caused between the first electrode-structures and the third electrode-structures and between the second electrode-structures and the third electrode-structures to generate an ionic wind that flows from the first electrode-structures and the second electrode-structures in a direction away from the third electrode-structures.

2. The air purifier according to claim 1, wherein an end of each of the third electrode-structures is shaped as a plane, the end being located toward the corresponding one of the second electrode-structures.

3. The air purifier according to claim 1, wherein a distance between the first conductive area of one of the first electrode-structures and a corresponding one of the third electrode-structures is longer than a distance between the second conductive area of a corresponding one of the second electrode-structures and the corresponding one of the third electrode-structures.

4. The air purifier according to claim 1, wherein a width of the coupling portion is smaller than a width of the first electrode portion and a width of the second electrode portion.

5. The air purifier according to claim 1,
wherein the first electrode-plate further comprises
a fourth electrode portion that is plate-like, the fourth electrode portion comprising a plurality of fourth electrode-structures, each of the fourth electrode-structures having a fifth hollow that is substantially shaped as a circle and is larger in diameter than the third hollow, and having a fourth conductive area that is a conductive area at a rim of the fifth hollow, the fourth electrode-structures being formed at locations corresponding to respective locations of the second electrode-structures; and
a second coupling portion coupling the fourth electrode portion and the second electrode portion and having a bent shape to place the fourth electrode portion vertically above the second electrode portion such that each of the fourth electrode-structures and a corresponding one of the second electrode-structures are concentric.

6. A production method for the air purifier of claim 1,
wherein the first electrode portion, the second electrode portion, and the coupling portion are formed in a conductive plate that is plate-like, by performing press working, and
bending is performed on the coupling portion of the conductive plate in which the first electrode portion, the second electrode portion, and the coupling portion have been formed, to produce the first electrode-plate.

7. The production method for the air purifier according to claim 6, wherein the third electrode-structures are formed in a conductive plate that is plate-like, by performing press working.

8. A production method for the air purifier of claim 5,
wherein the first electrode portion, the second electrode portion, the fourth electrode portion, the coupling portion, and the second coupling portion are formed in a conductive plate that is plate-like, by performing press working, and
bending is performed on the coupling portion and the second coupling portion of the conductive plate in which the first electrode portion, the second electrode portion, the fourth electrode portion, the coupling portion, and the second coupling portion have been formed, to produce the first electrode-plate.

9. The production method for the air purifier according to claim 8, wherein the third electrode-structures are formed in a conductive plate that is plate-like, by performing press working.
